(19) United States
(12) Patent Application Publication
(10) Pub. No.: US 2021/0267960 A1
(43) Pub. Date: Sep. 2, 2021

(54) METHODS OF TREATMENT OF INFLAMMATORY CYTOKINE-RELATED ARTHRITIC DISORDERS

(71) Applicant: Bridge Pharma Inc., Sarasota, FL (US)

(72) Inventors: A.K. Gunnar Aberg, Sarasota, FL (US); Vincent B. Ciofalo, Branford, CT (US); Kresimir Pucaj, Zagreb (HR)

(21) Appl. No.: 17/185,034

(22) Filed: Feb. 25, 2021

Related U.S. Application Data
(60) Provisional application No. 62/982,938, filed on Feb. 28, 2020.

(51) Int. Cl. A61K 31/4535; A61K 39/395; A61K 31/519; A61K 45/06; A61K 9/00; A61K 9/20

(52) U.S. Cl. CPC A61K 31/4535; A61K 39/3955; A61K 9/20; A61K 45/06; A61K 9/0053; A61K 31/519

(57) ABSTRACT

Methods of treating a patient suffering from a TNFα associated arthritic disorder with the cytokine release inhibitor norketotifen include orally or topically administering to the subject in need thereof a therapeutically effective amount of norketotifen, an isomeric mixture, a prodrug, or a pharmaceutically acceptable salt thereof. Also included are methods of intra-articular injection of norketotifen, such as into a joint of a subject in need thereof.

### CROSS-REFERENCE TO RELATED APPLICATIONS

This application claims priority to U.S. Provisional Application 62/982,938 filed on Feb. 28, 2020, which is incorporated herein by reference in its entirety.

### TECHNICAL FIELD

The aspects disclosed herein relate to methods for the treatment of disorders associated with the inflammatory cytokine TNFα and other pro-inflammatory cytokines with norketotifen. Norketotifen is a benzocycloheptathiophen molecule, related to ketotifen (Zaditen®, Zaditor®, Novartis), but with a non-substituted nitrogen atom. Contrary to ketotifen, norketotifen has now been found to potently inhibit the release of multiple inflammatory cytokines and chemokines from pro-inflammatory cells.

### BACKGROUND

Tumor Necrosis Factor α (TNFα or just TNF) is a cytokine produced by numerous pro-inflammatory cell types and released from these cells upon activation. TNFα is known to activate two receptors, called TNFR1 and TNFR2. TNFα has been implicated in the pathophysiology of a variety of inflammatory diseases and disorders, including inflammatory arthritic diseases.

Because of the harmful role of human TNFα in a variety of human disorders, therapeutic strategies have been designed to inhibit or counteract hTNFα activity. Most of the marketed TNFα blockers such as Humira® (adalimumab) are monoclonal antibodies. Adalimumab and other monoclonal antibodies are administered as injections and have been approved for the treatment of numerous TNFα related diseases, such as for example arthritic diseases such as osteoarthritis, rheumatoid arthritis, juvenile idiopathic arthritis, psoriatic arthritis and ankylosing spondylitis. While effective, monoclonal antibody therapies are extremely expensive and must be administered by injection. What is needed are small molecule TNFα blockers, preferably TNFα blockers that can be orally administered.

### SUMMARY

In an aspect, a method of treating a TNFα associated disorder, such as specific arthritic diseases, in a subject in need of such treatment comprises orally or topically administering to the subject in need thereof a therapeutically effective amount of the TNFα inhibitor norketotifen, an isomer, a prodrug, or a pharmaceutically acceptable salt thereof.

In another aspect, a method of treating a TNFα associated arthritic disorder in a subject in need of such treatment comprises intra-articularly injecting to the subject in need thereof a therapeutically effective amount of norketotifen, an isomer, an isomeric mixture, or a pharmaceutically acceptable salt thereof.

In an aspect it has now been found that the release of TNFα and other pro-inflammatory cytokines from pro-inflammatory cells are potently and dose-dependently inhibited by norketotifen but not by a chemically closely related reference compound, called ketotifen.

### DETAILED DESCRIPTION

Described herein are studies showing that norketotifen is unexpectedly a highly effective inhibitor of TNFα, and other cytokines, such as for example IL-5, IL-4 and IL-13. Norketotifen is thus particularly useful in the treatment of diseases treatable with the TNFα blocking agents adalimumab (Humira®), infliximab (Remicade®, Inflectra®), certolizumab pegol (Cimzia®), golimumab (Simponi®) and enteracept (Enbrel®); the IL-4 and IL-13 inhibitors dupilumab (Dupixent®), pascolizumab, pitrakinra, and lebrikizumab, anrukinzumab, and tralokinumab; and the IL-5 inhibitors mepolizumab, reslizumab and benralizumab. Advantageously, norketotifen is a small molecule and oral and parenteral administration of norketotifen can provide for convenience and significant cost savings. In addition, norketotifen was found in preclinical studies to be free from the adverse immune-suppressive effects of the steroids.

The term TNFα as used herein refers to human, feline, canine or equine TNFα. The human cytokine TNFα is a 17 kD secreted form and a 26 kD membrane associated form, the biologically active form of which is composed of a trimer of noncovalently bound 17 kD molecules. The chemical structure of hTNFα is described in the art. The human TNFα can be prepared by standard recombinant expression methods or can be purchased commercially.

The term “subject” is intended to include living organisms. Examples of subjects include mammals, e.g., humans, dogs, cows, horses, pigs, sheep, goats, cats, mice, rabbits, rats, and transgenic non-human animals. Specific subjects are humans, dogs and horses.

As used herein, the term norketotifen refers to racemic norketotifen free base, an isomer, an isomeric mixture, a prodrug, or a pharmaceutically acceptable salt thereof. RS-norketotifen refers to racemic norketotifen. In an aspect, the compound is RS-norketotifen free base, RS-norketotifen hydrogen fumarate or RS-norketotifen hydrochloride.

Norketotifen is an achiral molecule, but has two atropisomers, S-norketotifen and R-norketotifen, as has previously been described by Aberg et al. in U.S. Pat. Nos. 7,226,934 and 7,557,128.

As explained in U.S. Pat. Nos. 7,226,934 and 7,557,128, norketotifen had significant sedative effects when studied in an art-accepted mouse model of sedation, and further, the sedative effects were attributed to the R-isomer. It was thus proposed that only the S-isomer could be administered without sedative side effects. It has later been found that orally administered RS-norketotifen is free from sedative side effects in dogs (U.S. Pat. No. 8,557,846) and in humans (U.S. Pat. Nos. 9,138,431 and 9,345,697). Therefore, unlike for ketotifen, no dose-limiting sedative adverse effects are expected for norketotifen, even after high oral doses of norketotifen to non-rodent species.

Norketotifen can be made using methods known in the art, as described in U.S. Pat. No. 3,682,930, the disclosure of which is hereby incorporated by reference for its teaching of the synthesis of norketotifen.

Prodrugs are molecules that can be metabolized to provide an active compound, in this case norketotifen. Prodrugs of norketotifen include N-substituted hydroxyalkyl or carboxyalkyloxyalkyl analogs thereof. Such molecules are described in U.S. Pat. No. 6,297,683. Prodrugs of norketotifen include molecules of the formula:

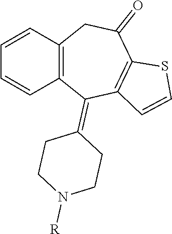

wherein R is alkyl or hydroxy alkyl or carboxy-alkoxy-alkyl. Additional prodrugs include substituents at the 8-position and/or the 10-position and/or inclusion of substituents on various positions on the piperidine ring of the molecule shown above.

As used herein, the terms “pharmaceutically acceptable salts” or “a pharmaceutically acceptable salt thereof” refer to norketotifen salts, which have been prepared from pharmaceutically acceptable non-toxic acids. Exemplary pharmaceutically acceptable acid salts include acetic, benzenesulfonic (besylate), benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pathothenic, phosphoric, p-toluenesulfonic, succinic, sulfuric, tartaric, and the like. The hydrochloride salt and the hydrogen fumarate salt are particularly preferred.

In a pharmacological sense, a “therapeutically effective amount” or “effective amount” of norketotifen refers to an amount effective in the prevention or treatment of a disorder for the treatment of which norketotifen is effective. The term “disorder” as used herein is synonymous to the term “disease” or a condition that would benefit from treatment with norketotifen. This includes chronic and acute disorders or diseases including those pathological conditions which predisposes the subject to the disorder in question.

In an aspect, a method of treating a TNFα associated disorder in a subject in need of such treatment comprises administering, particularly orally or topically administering, to the subject in need thereof a therapeutically effective amount of norketotifen, an isomer, a prodrug, or a pharmaceutically acceptable salt thereof.

As used herein, the term “TNFα associated disorder” or “a disorder in which TNFα activity is detrimental” is intended to include diseases and other disorders in which the presence of TNFα in a subject suffering from the disorder has been shown to be or is suspected of being either responsible for the pathophysiology of the disorder or a factor that contributes to a worsening of the disorder. Accordingly, a TNFα associated disorder is a disorder in which inhibition of TNFα activity alleviates the disorder and/or the symptoms of the disorder. Such disorders may be evidenced, for example, by an increase in the concentration of TNFα and possibly other cytokines in a biological fluid from a subject suffering from the disorder (e.g., an increase in the concentration of TNFα and additional cytokines in serum, plasma, synovial fluid, etc.) of the patient.

TNFα has been implicated in playing a role in the pathophysiology of a variety of autoimmune diseases. For example, TNFα has been implicated in activating tissue inflammation and causing joint destruction in patients suffering from osteoarthritis.

Arthritis is an umbrella term that is used to describe inflammation of the joints of mammals. However, there are different kinds of arthritis, including the wide-spread diseases rheumatoid arthritis (RA) and osteoarthritis (OA). RA is an autoimmune disorder while OA is a degenerative joint condition, however, both forms of arthritis are TNFα associated disorders.

Rheumatoid Arthritis (RA) is a TNFα associated autoimmune disease that causes chronic inflammation and pain in the joints and other areas of the body. RA that starts before the age of 16 is often referred to as Juvenile Idiopathic Arthritis but is for simplicity herein is included with Rheumatoid Arthritis. The involvement of pro-inflammatory cytokines TNFα, IL-1 and IL-6 in both human and canine RA are well known. Human synovial fluid also contains elevated concentrations of the chemokines IL-8, MIP-1α and MCP-1. It has now been found that norketotifen potently inhibits the release not only of TNFα, but also of IL-1, IL-6, IL-8, MIP-1α and MCP-1 (Table 1). It is therefore believed that norketotifen will be of therapeutic value for human and canine patients suffering from RA or juvenile idiopathic arthritis. In addition to reducing RA, norketotifen is expected to reduce symptoms of RA, such as for example joint pain, swelling and stiffness, fatigue, muscle pain, and worsening joint stiffness after sleep or prolonged stillness. RA is a common disease in dogs, particularly in older animals.

Osteoarthritis (OA) is the most common form of arthritis in humans and is a degenerative joint disorder, expressing breakdown of the cartilage that cushions the joints. The wearing down of cartilage causes the bones to rub against each other, which exposes small nerve endings and explains pain. OA does not involve an autoimmune process, but similar to RA, the disorder OA will often also express inflammation, which often is milder in OA patients than in RA patients. OA most commonly affects the hands, knees, hips, lower back, and neck. OA is a common disease in dogs and horses, particularly in older animals.

Two cytokines are well known to be involved in OA: TNFα and IL-1beta and contrary to the selective TNFα monoclonal antibodies that have been tested in OA-patients, norketotifen inhibits the release of both TNFα and IL-1beta (Table 1). Published studies have demonstrated that IL-1beta is involved in the breakdown of cartilage but to our knowledge few studies have been design with combined inhibition of TNFα and IL-1beta. Apart from TNFα and IL-1beta, other cytokines including IL-6 and the chemokine IL-8 have also been shown to be implicated in OA. Theoretically, combination-therapy, like that offered by norketotifen may have significant advantages over the use of TNF-selective antibodies that are only targeting TNFα. Also from studies of horses with OA, increased synovial concentrations of TNFα, INFγ, IL-1β, IL-6 and IL-10 were reported.

By inhibiting multiple cytokines and chemokines, norketotifen can also reduce the signs and symptoms of moderate to severe polyarticular juvenile idiopathic arthritis in children. In an aspect, norketotifen can be combined with other medications for arthritis, such as methotrexate and/or TNFα-selective monoclonal antibodies, and/or corticosteroids.

Psoriatic arthritis (PsA), is another TNFα associated arthritic disease and is expressing inflammation of the joints, causing pain and stiffness. PsA can be accompanied by psoriasis, an itchy and painful skin condition. PsA generally affects the large joints, such as those in the lower extremities and distal joints of the fingers and toes, but can also affect the back and the sacroiliac joints of the pelvis. Norketotifen is expected reduce inflammation and pain, like the TNFα neutralizer etanercept and may have additional therapeutic effects on PsA by inhibiting cytokines such as IL-10, IL-13, INFγ and MIP-1α and may prevent further damage to the bones and joints, and may help the PsA patients to better perform their daily activities.

Ankylosing spondylitis (AS) is another TNFα associated arthritic disease that over time can cause the spinal vertebrae to fuse. Early signs and symptoms of ankylosing spondylitis include pain and stiffness in the lower back and hips, particularly in the morning and after periods of inactivity. Neck pain and fatigue also are commonly observed. In a meta-analysis, the results in multiple studies of the TNF-inhibitor were compared—the results were not impressive and the most active drug was etanercept which is not a monoclonal antibody. The effects of other cytokines on AS have been studied and it was concluded that not only TNFα but several other cytokines, such as for example IL-10 are involved in AS. Since norketotifen inhibits TNFα and numerous additional cytokines, it is likely that norketotifen is a preferred medication when compared with selective TNFα inhibitors.

Plaque psoriasis is an inflammatory joint and skin condition that is localized in joints and to specific dermal areas, called plaques that are surrounded by healthy skin. Thus, the disease called plaque psoriasis is clinically vastly different from classic psoriasis. The plaques are most often red with white scales. The plaques are most often pruritic and can therefore be called itchy plaques. Classic psoriasis is not localized to distinct patches which is a hallmark of plaque psoriasis. The cytokines involved in plaque psoriasis are fewer than the cytokines involved in classic psoriasis, but the large numbers of cytokines involved makes it difficult to determine if the differences between classic psoriasis abs plaque psoriasis are due to one or more specific cytokines. Plaque psoriasis responds to TNFα-inhibitors, but it may be difficult to defend the use of expensive systemic medication for patients with a limited number of psoriatic plaques. Therefore, plaque psoriasis may be treated with topically formulated norketotifen. Also, it is known that plaque psoriasis expresses numerous cytokines both in the inflamed joints and in the inflamed dermal plaques, while monoclonal antibodies are usually selectively inhibiting a single cytokine. Thus, for example, the IL-1 family and the IL-10 family of cytokines are known to be involved in plaque psoriasis. Members of those cytokine families have now been tested and have been found to be potently inhibited by norketotifen. Other cytokine families may also be expressed by patients suffering from plaque psoriasis but their sensitivity to norketotifen remains to be tested. Relying on the currently available test results with TNFα and members of the IL-1 and IL-10 families of cytokines (TABLE 1) it is now believed that norketotifen will have therapeutic advantages over THFα-selective monoclonal antibodies. It is reasonable to believe that norketotifen will be dermally active after application topically on the inflamed and itchy plaques of patients suffering from plaque psoriasis.

Hidradenitis suppurativa (HS) is also is a TNFα associated chronic inflammatory condition. HS causes small, painful lumps to form under the skin. The lumps can break open, or tunnels can form under the skin. The condition mostly affects areas where the skin rubs together, such as the armpits, groin, buttocks and breasts. Autoimmune conditions have been shown to be associated with HS including inflammatory bowel disease. Initial evidence for the use of TNF-α inhibitors in HS stemmed from recognition that inflammatory bowel disease patients treated with these medications saw a concurrent improvement in their HS symptoms. Norketotifen can reduce the inflammatory nodules and abscesses associated with HS. HS may be treated with topical (dermal) or with oral administration of norketotifen

In another aspect, the methods can further comprise co-administration of orally or topically administered norketotifen with orally, parenterally or dermally administered drugs that may reinforce or complement doses of norketotifen. Norketotifen can also be combined with one or more monoclonal antibodies or proteins that blocks TNFα, IL-5, IL-4, IL-13, IL-5, IL-4 and/or IL-13, or any monoclonal antibodies that typically will administrated by injection. Exemplary monoclonal antibodies or proteins that may be combined with norketotifen comprise adalimumab, infliximab, certolizumab pegol, golimumab, dupilumab, pascolizumab, enteracept, pitrakinra, lebrikizumab, anrukinzumab, tralokinumab, mepolizumab, reslizumab, benralizumab, and combinations thereof.

The term “pharmaceutical formulation” or “pharmaceutical composition” refers to preparations which are in such form as to permit the biological activity of the active ingredients to be unequivocally effective, and which contain no additional components which are significantly toxic to the subjects to which the formulation would be administered. “Pharmaceutically acceptable” excipients (vehicles, additives) are those which can reasonably be administered to a subject mammal to provide an effective dose of the active ingredient employed.

The embodiments disclosed herein also provide pharmaceutical compositions, which comprise norketotifen, formulated together with one or more pharmaceutically acceptable excipients.

Pharmaceutical compositions for oral administration of solid dosage forms include capsules, tablets and liquid dosage forms. In solid dosage forms, the active compound may be mixed with one or more pharmaceutically acceptable excipients or carriers (such as for example sodium citrate, dicalcium phosphate), fillers or extenders (such as for example starch, lactose, sucrose, glucose, mannitol, silicic acid), binders (such as for example alginates, carboxymethylcellulose, gelatin, polyvinylpyrrolidone, sucrose, acacia), humectants (such as for example glycerol), solution retarding agents (such as for example paraffin), disintegrating agents (such as for example agar-agar, calcium carbonate, starch, alginic acid, silicates, sodium carbonate), absorption accelerators (such as for example quaternary ammonium compounds), wetting agents (such as for example cetyl alcohol, glycerol monostearate), absorbents (such as for example kaolin, bentonite clay), lubricating agents (such as for example talc, calcium stearate, magnesium stearate, polyethylene glycols, sodium lauryl sulfate), and/or other excipients, such as for example buffering agents. Solid forms of capsules, granules, pills, and tablets can have coatings and/or shells (such as for example enteric coatings) known in the art. The compositions may also be designed to release the active ingredient(s) in a certain part of the gastrointestinal tract or in a controlled release, slow-release or in a delayed-release manner. The active compound(s) can also be microencapsulated with one or more of the above-mentioned excipients or other suitable excipients. Liquid dosage forms for oral administration of norketotifen include pharmaceutically acceptable emulsions, solutions, suspensions, syrups and elixirs. The liquid dosage form may also contain commonly known diluents (such as for example water, other solvents, solubilizing agents), emulsifiers, such as for example ethanol, isopropyl alcohol, ethyl carbonate, ethyl acetate, benzyl alcohol, benzyl benzoate, propylene glycol, butylene glycol, dimethyl formamide, oils, oleic acid, glycerol, polyethylene glycols, sorbitan fatty esters, and mixtures thereof.

The actual dosage levels of active ingredients in the pharmaceutical compositions disclosed herein may be varied so as to obtain the desired therapeutic effect. Thus, the amount of drug used varies and will depend on factors such as the administration form, the severity of the disease, the frequency of dosing, and other circumstances (such as general health, body weight, age, etc.) known to the patient, the caretaker of the patient and/or the caring physician.

The therapeutically effective oral doses of norketotifen useful for treating human patients will be determined by the caring physician and are generally 0.5 mg to 50 mg (calculated as norketotifen free base), dosed orally as the free base or as a salt, such as for example the hydrochloride or mesylate salts or the hydrogen fumarate salt, once, twice or more times daily. In one embodiment, the treatment is once daily dosing. The therapeutically effective dose may be administered less than once daily, such as for example one to six times weekly, wherein administration is over at least a one-week period, as determined by the patient, the caretaker of the patient and/or the caring physician.

As an alternative or in addition to oral administration, norketotifen can be effective when administered topically or transdermally.

In an aspect, a method of treating a TNFα associated arthritic disorder in a subject in need of such treatment comprises intra-articularly injecting to the subject in need thereof a therapeutically effective amount of norketotifen, an isomer, an isomeric mixture, or a pharmaceutically acceptable salt thereof Intra-articular injection can be into a joint such as the knee. For example, norketotifen can be administered as an intra-articular injection to a patient such as an OA patient who has demonstrated resistance to orally administered medications. Therapeutically administrated intra-articular norketotifen may be in the forms of solutions or suspensions. Since rapid and substantial efflux of NK from the joints are expected when standard crystalline solutions are used, extended release formulations are developed and may be preferred. Poly-lactic-to-glycolic acid (PLGA) biodegradable controlled drug delivery carriers have been described and may be used as a preferred. The total dose of NK to be injected into a joint such as a knee varies from about 1 milligram to 100 milligrams once monthly or more often as needed. The patient may be a human, a dog or a horse.

The invention is further illustrated by the following non-limiting examples.

### EXAMPLES

### Example 1

### Cytokine Inhibition by Norketotifen Objective

The objective of this study was to assess the ability of norketotifen to inhibit induced cytokine release from stimulated human white blood cells.

### Methodology

The study was conducted in two phases. The first phase included optimization of the test methodology and including dose-range finding studies, using buffy coats from 3 human donors, stimulated with phytohemagglutinin (PHA). Following optimization of the test conditions, a final protocol was issued. In the following Main Study, white blood cells (buffy coats) from 5 healthy human donors were stimulated with PHA, 5 μg/ml. The concentrations of the test article and the duration of exposure were optimized to obtain acceptable dose-response ratios. Thus, the inhibitory effects of three concentrations of NK and one concentration of ketotifen were evaluated in the Main Study. The buffy coats containing white blood cells from 5 male human donors were resuspended in assay media and were pre-incubated for 30 min with either vehicle/saline, ketotifen 10 μM (4.28 μg/ml) or norketotifen 1 μM and 10 μM and 100 μM, corresponding to 0.411 μg/ml, 4.11 μg/ml and 41.1 μg/ml, resp. Following these preincubations, the white blood cells were stimulated for 16 hours with PHA, 5 μg/ml. The cell system supernatants were then harvested and stored frozen pending analyses.

### Results

The following Table 1 demonstrates the lack of inhibition of cytokine and chemokine release by ketotifen, and the potent and dose-depending inhibitory effects of norketotifen on the in vitro release of the pro-inflammatory cytokines such as TNFα, IL-4 and IL-13 and chemokines, such as the examples IL-8, MCP-1 and MIP-1a.

TABLE 1
Inhibition by norketotifen and ketotifen
of PHA- induced release of cytokines.

| | | |
|---|---|---|
| Cytokine or | Ketotifen (%) | Norketotifen (%) |

| Chemokine | 10 uM | 1 uM | 10 uM | 100 uM |
|---|---|---|---|---|
| IL-6 | 0.00 | 0.00 | 33.13¹ | 88.16¹ |
| TNF-α | 0.00 | 4.54 | 42.19² | 89.13¹ |
| IL-4 | 0.00 | 0.00 | 14.32¹ | 98.27¹ |
| IL-10 | 0.00 | 12.71² | 24.80² | 90.40¹ |
| IL-2 | 0.00 | 0.00 | 46.63¹ | 93.47¹ |
| IL-1β | 0.00 | 0.00 | 16.20² | 57.00¹ |
| INF-γ | 0.00 | 0.00 | 16.54¹ | 71.56¹ |
| IL-8 | 19.99 | 10.29² | 34.36¹ | 78.29¹ |
| IL-13 | 0.00 | 0.00 | 34.49¹ | 74.25¹ |
| MCP-1 | N/A | 2.04 | 14.35² | 84.52¹ |
| MIP-1a | N/A | 9.53 | 25.94² | 90.99¹ |

Values marked ¹ denote a statistically significant difference (p < 0.05) when compared with vehicle.
Values marked ² denote decrease in concentration that was not statistically significant.

### Conclusions

Norketotifen potently and dose-dependently inhibited the release of cytokines from human white blood cells. No statistically significant inhibition of cytokine release was expressed by ketotifen in this study.

### Footnote

The concentration of the test article had to be high in this study since the test kit used a high concentration of the agonist PHA (5 μg/ml) and a long exposure time (16 hrs.).

### Example 2

### Exemplary Oral Dosage Formulation

To make tablets, NK was blended with lactose and cellulose until a uniform blend is formed. The blue lake was added and further blended. Finally, the calcium stearate is blended in, and the resulting mixture is compressed into tablets using for example a 9/32-inch (7 mm) shallow concave punch. Tablets of other strengths may be prepared by altering the ratio of active ingredient to the excipients or to the final weight of the tablet. Those skilled in the art realize that formulations can also be administered to the patient in the form of for example a capsule, a cream, an ointment or a liquid formulation. Both norketotifen salts and norketotifen free base can be formulated as tablets.

TABLE 2
Tablet formulations

| Ingredient | Amount per tablet | | Amount per batch | |
|---|---|---|---|---|
| Norketotifen (NK) | 8 | mg | 800 | g |
| Microcrystalline cellulose | 24 | mg | 2400 | g |
| Lactose | 56 | mg | 5600 | g |
| Calcium stearate | 1.4 | mg | 140 | g |
| FD&C Blue #1 Lake | 0.03 | mg | 3 | g |

As used herein, the term “prodrug” refers to a medication or a compound that, after administration is metabolized into a pharmacologically active drug.

As used herein, the terms “pharmaceutically acceptable salts” or “a pharmaceutically acceptable salt thereof” refer to norketotifen salts, which have been prepared from pharmaceutically acceptable non-toxic acids. Exemplary pharmaceutically acceptable acid as for the compound of the present invention include acetic, benzenesulfonic (besylate), benzoic, camphorsulfonic, citric, ethanesulfonic, fumaric, gluconic, glutamic, hydrobromic, hydrogen fumaric, hydrochloric, isethionic, lactic, maleic, malic, mandelic, methanesulfonic, mucic, nitric, pamoic, pathothenic, phosphoric, p-toluenesulfonic, succinic, sulfuric, tartaric, and the like. The hydrochloride salt and the hydrogen fumarate salt are particularly preferred.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted.

As used herein, when referring to dosage amount, the term “about” includes amounts to ±10% of the recited value.

As used herein, the term “chronic administration” is defined as three or more consecutive days of administration. Acute administration of norketotifen refers to a single administration of the drug.

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

1. A method of treating a TNFα associated arthritic disorder in a subject in need of such treatment, the method comprising orally or topically administering to the subject in need thereof a therapeutically effective amount of norketotifen, an isomer, an isomeric mixture, a prodrug, or a pharmaceutically acceptable salt thereof.

2. The method of claim 1, wherein the subject is a human subject, a dog, or a horse.

3. The method of claim 1, wherein the arthritic disorder is rheumatoid arthritis, osteoarthritis, juvenile idiopathic arthritis, psoriatic arthritis, or ankylosing spondylitis.

4. The method of claim 1, further comprising co-administering with norketotifen a monoclonal antibody or protein that specifically binds TNFα, IL-5, IL-4 and/or IL-13.

5. The method of claim 1, further comprising co-administering methotrexate with the norketotifen.

6. The method of claim 1, further comprising co-administering a corticosteroid with the norketotifen.

7. The method of claim 1, wherein the norketotifen is orally administered in the form of a tablet, a capsule, or a syrup.

8. The method of claim 1, wherein the therapeutically effective amount of norketotifen is from about 1 mg to about 30 mg of racemic norketotifen, an isomer, a prodrug or a pharmaceutically acceptable salt thereof, calculated as norketotifen free base and administered one or more times daily.

9. A method of treating a TNFα associated arthritic disorder in a subject in need of such treatment, the method comprising intra-articularly injecting to the subject in need thereof a therapeutically effective amount of norketotifen, an isomer, an isomeric mixture, or a pharmaceutically acceptable salt thereof.

10. The method of claim 9, wherein the subject is an osteoarthritis patient with a demonstrated resistance to orally administered norketotifen.

11. The method of claim 9, wherein injecting is performed into a joint of the subject.

12. The method of claim 9, wherein the subject is a human, a dog or a horse.
